(19) United States
(12) Patent Application Publication
(10) Pub. No.: US 2007/0073174 A1
(43) Pub. Date: Mar. 29, 2007

(54) Sphygmomanometer adaptable to limbs of different measurements

(76) Inventor: Ricardo Rodriguez Aranciva, San Luis (AR)

Correspondence Address: PAUL L. BROWN;AMIN HALLIHAN LLC, 217 N. JEFFERSON ST., FIRST FLOOR, CHICAGO, IL, 60661, US

(21) Appl. No.: 11/222,617

(22) Filed: Sep. 9, 2005

(30) Foreign Application Priority Data
Sep. 9, 2004 (AR) P040103230
Apr. 15, 2005 (AR) P050101482

(51) Int. Cl. A61B 5/02

(52) U.S. Cl. 600/499; 600/490

(57) ABSTRACT

A sphygmomanometer device for measuring the blood pressure of all ages of patients including a flexible band having a plurality of insufflable chambers, with each chamber structurally arranged to communicate with an insufflation pump. The insufflable chambers are sized to the age and physical condition of the patient when the flexible band is applied about the patents arms or legs to determine the patients blood pressure.

### REFERENCE TO RELATED FOREIGN APPLICATIONS

This application is related to and claims the benefit under Title 35, United States Code, §119 of Argentina Application No. P040103230, filed Sep. 9, 2004, and Argentina Application No. P050101482, filed Jul. 15, 2005, the teachings of which are incorporated herein by reference.

### BACKGROUND OF THE INVENTION

This invention refers to a sphygmomanometer which is adaptable to limbs—arms, legs—of different measurements. More particularly, a multi-purpose sphygmomanometer for measuring blood pressure in patients of different ages and physical build, wherefor the manner of usage of this sphygmomanometer may be adapted following various options such as, for instance, adults and children, obese adults and standard size adults, children and neonates. These options are possible thanks to the special construction features of the sphygmomanometer, designed specifically to attain efficiently said purpose.

Within the previous art in the subject, several types of sphygmomanometers are known, starting with traditional aneroid mercury sphygmomanometers, and up to the most modern designs which use last-generation technology, such as automatic digital sphygmomanometers with electronic memory for recording data pertaining to measurements carried out. With devices of high technological complexity, both those working in conjunction and those used for out-patients, measurements of blood pressure can be taken as well as measurements of other vital signs such as heart rate, breathing rate, etc. Beyond any doubt, the latter include worthy operational features, with useful characteristics and their use is very simple both for professionals and directly for the patients themselves. However, it is not less true that in the above cases, i.e., in mercury, aneroid or electronic technology sphygmomanometers, the need has not been foreseen to adapt them conveniently in order to be able to use them in adults as in children, and even less neonates selectively with a single device.

It is, then, with a view to overcome the limitations of the aforementioned sphygmomanometers, that the one which is the object of this invention has been developed. In fact, thanks to its outstanding features, this multipurpose sphygmomanometer is adequate for measuring the blood pressure both of adults and children and neonates, both obese and normal, according to such combinations as may prove necessary, with a recording system for all ages and in accordance with varying nutritional conditions. As it will be explained below with reference to the figures which illustrate this sphygmomanometer, for the correct measurement of the blood pressure of each type of patient in particular, it is enough to interchange, super-pose and/or introsuscept (to arrange one inside the other) insufflable chambers, either individual or in compartments and to select the insufflation path as required in each case.

It is, therefore, the object of this invention, to provide a multipurpose sphygmomanometer for measuring blood pressure in adults, children and neonates, which comprises a clamping band which defines a bracelet, a wrist protector, a wristlet, a leg guard or equivalent to surround the part of the patient anatomy from where the blood pressure is to be measured, with a set of insufflable and selectable chambers, which can be combined one with the other as required, attached to said clamping band, in order to measure the blood pressure of adults, children and neonates, in all cases regardless of their weight and physical condition. The chambers are connected to means of recording blood pressure, to an insufflation pump and to an air-actuated switch or selector device to select the chamber or chambers to be insufflated, individually or simultaneously, depending on the type of patient that is to be handled. According to the various embodiment alter-natives, the chambers may be, for instance, overlapping and at least in part removable one with respect to the others and the clamping band, cleared and isolated from one another by means of a flexible material, arranged one inside the other and/or divided in compartments separated by partitions.

In one of its forms of embodiment, this sphygmomanometer comprises a selector device which can include one or more inlets which can be connected to one or more outlets, it being possible, in addition, to change the direction of fluid circulation using each inlet as an outlet and vice versa.

The bidirectional selection and/or combination of inlets and outlets in order to define fluid circulation paths may be made, for example, among:
inlet and an outlet;
an inlet and several outlets;
several inlets and an outlet;
several inlets and several outlets;

The device drive may be of various types, according to different manufacture features. In fact, according to its type of drive, the switch may be, for example:
Manual: rotating or with linear movement.
Automatic: electrical, electromechanical, electromagnetic, air-actuated or hydraulic (as an option with remote actuation and control).

As it has been mentioned already, one of the preferred uses of this device is to control the injection of air in a multipurpose tensiometer for neonates, children and adults of varying ages and physical builds. To this end, it will be mounted in an appropriate support according to the embodiment involved and the inflatable chambers to be used.

This switch may be build in different forms and with different materials, such as, for instance, metals, PVC, Teflon, plastic materials in general, rubbers, etc., and any combination thereof, in accordance with the needs of usage.

### DESCRIPTION OF THE DRAWINGS

FIG. 1 is a representative diagram of the sphygmomanometer object of this invention;

FIG. 2 is a representative diagram of a selector device of the corresponding chamber to be insufflated according to an alternative embodiment of the illustration in FIG. 1;

FIG. 3 is a perspective view which shows an alternative embodiment of the selector device of the chamber to be insufflated applied to the sphygmomanometer;

FIG. 4 shows the selector device in FIG. 3 now as an integral part of the assembly which includes, as alternative embodiment, the insufflation elements and elements for recording blood pressure;

FIG. 5 is a representative diagram of one alternative embodiment of the sphygmomanometer which is described, where in this case three chambers to be insufflated are included, each to be used respectively according to the size and age of the patient; and

FIG. 6 illustrate the manner in which the arm of a patient is wrapped with the appropriate chamber and then adjusted with a clamping band or bracelet for the purpose of measuring his blood pressure; and

FIG. 7 is a perspective view of the device according to one of the preferred embodiments to be put into practice;

FIGS. 8 to 10 are representative diagrams of a cross section of a basic or primary embodiment of the device object of this invention;

FIG. 11 to 14 are representative diagrams of a cross section of a more elaborate alternative embodiment of the device in FIGS. 8 to 10;

FIG. 15 to 18 are representative diagrams of a cross section of an alternative embodiment shown in FIGS. 11 to 14;

FIG. 19 is an elevated view in partial cross section of the device according to the embodiment of FIG. 7, where several transverse planes are shown corresponding to the illustrations in FIGS. 20 to 23;

FIG. 20 to 23 are representative diagrams of the transverse cuts shown in FIG. 5;

### DETAILED DESCRIPTION OF THE INVENTION

Firstly, with reference to the diagram of the sphygmomanometer in FIG. 1, identified with general reference number 1, a clamping band 2 can be seen, which defines a bracelet, a wrist protector, a wristlet, a leg guard or equivalent, which includes tissue 3a and 3b of the “thistle” type, the latter attached to the obverse of band 3, for adjusting same on the anatomical part of the patient from where his blood pressure will be measured. In practice, band 2 may be optionally made of a rigid, flexible or elastic material, of tissue or cloth of any type, as deemed more appropriate, preferably with standard attachment fittings.

According to this first embodiment, on band 2 a set of insufflable chambers is arranged, which include a chamber 4 required to measure blood pressure in adults and a chamber 5 required to measure blood pressure in children as well as in neonates or for general pediatric use. As shown, chambers 4 and 5 overlap one with the other, are removable at least in part one from the other and from the band or bracelet 2 and each one of them includes an extension positively related to the size and age of the patient, chamber 4 (for adults) being used for larger widths and lengths than chamber 5 (for children or neonates).

According to the alternative embodiments foreseen for sphygmomanometer 1, chambers 4 and 5 may be fixed in whole or in part and removable one from the other and from the surface of band 2, in whole or in part, divided inside in compartments by means of laminated flexible material or by means of transverse or longitudinal, or introsuspected partitions, i.e., arranged one inside of the other.

The set comprised of chambers 4 and 5 is connected to an insufflation pump, defined by a rubber knob 6 with an air discharge cock 6, through respective air conduits 7a and 7b for chamber 4, 8a and 8b for chamber 5, and a selector device by means of which air is channeled to one sleeve or the other as required. Said conduits 7a and 7b, or 7A to 7c, may be parts of separate paths, as illustrated, or may be part of a single hose or “multi-lumen”. The same is valid for conduits 8a and 8b.

Selector device 9 is defined by an air-actuated selector switch 9 which, in this first embodiment, is a two-way rotary switch actuated by a knob 10, or else, as it will be seen below, a linear lever-actuated switch. Depending on the number and configuration of the chambers, switch 9 may also be a three- or two-way device. Selector device 9 is provided with an air inlet connected to knob 6 by means of conduit 7c and a device to record or read blood pressure, in this case of the aneroid or manometer type identified with reference number 11 by means of conduit 8c.

It should be pointed out that to read blood pressure in practice a recording device could likewise be used, of the mercury column or digital electronic type, among other possible types, as it may be deemed more appropriate eventually. In addition, both the recording means and the insufflation means may be portable, with a mobile or wall-mounted support.

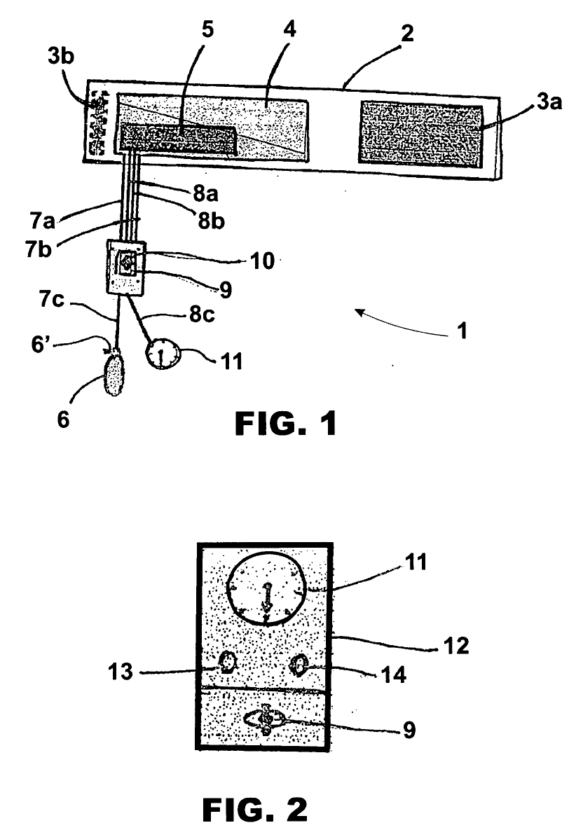

FIG. 2 shows schematically an alternative embodiment where se-lector switch 9 is housed in a receptacle, identified with reference number 12, together with manometer 11 and an automatic insufflation pump (not shown) which may be, for example, electronic or electromechanical, among other possibilities. In this case, in receptacle 12, an insufflation push-button 13 and an air-discharge push-button 14 are included, related from the operational standpoint to the air pump housed therein.

In the embodiment variant shown in FIG. 3, it is clear that the selector device in the chamber to be insufflated, applied to the sphygmomanometer and, in particular, the selector switch identified now with reference number 30, is of the type actuated by means of a linear lever 31. This se-lector switch 30 may be based on an operating principle similar to that of switch 9 of the previous embodiment and, in general, may be any type of air-channeling path selector switch for various possible applications. As shown, the connections of selector switch 30 with rubber knob 6 and manometer 11 through conduits 7c and 8c, on the one hand, and the connections for the corresponding chambers through the “single-lumen” or “multi-lumen” conduits 7a, 7b, 8a and 8b, on the other, are the same as those illustrated in FIG. 1.

Depending on the number of chambers associated with clamping band 2, switch 9 and switch 30 will be, for example, six-way (two inlets and four selective outlets), or eight-way (two inlets and six selective outlets).

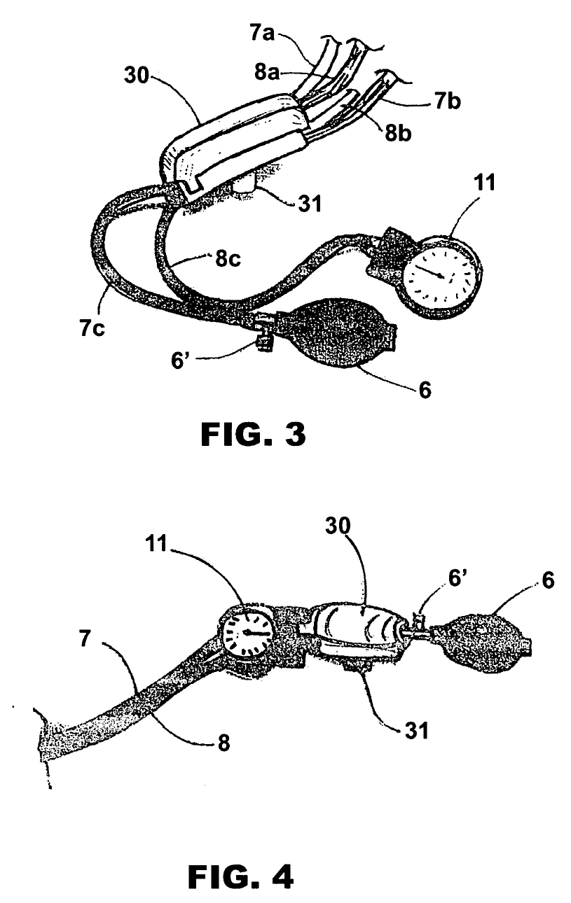

In FIG. 4, another alternative embodiment is shown, where selector switch 30 is connected as an integral part of an assembly where it inter-faces with the same rubber knob 6 and manometer 11, i.e., without conduits 7c and 8c, while the chambers to be insufflated, as before by means of the conduit sets identified by references 7 and 8, in this case of the “multi-lumen” type, although they could be individual conduits of the “single-lumen” type. This alternative embodiment is, thus, a compact assembly which can be handled with one hand.

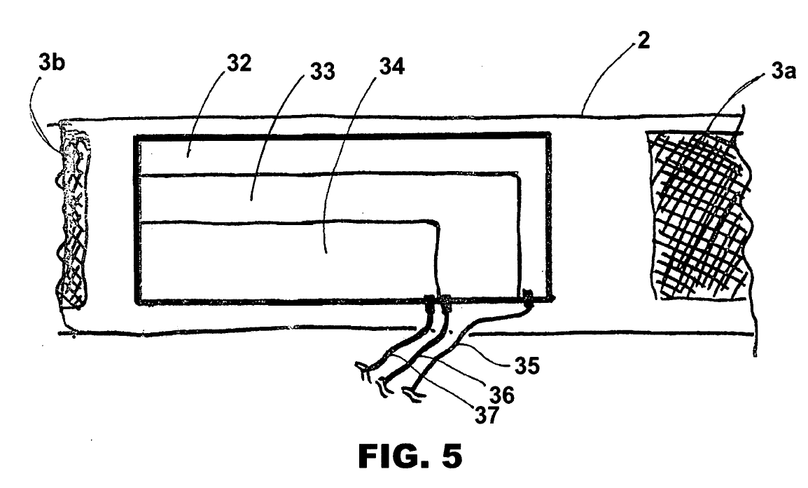

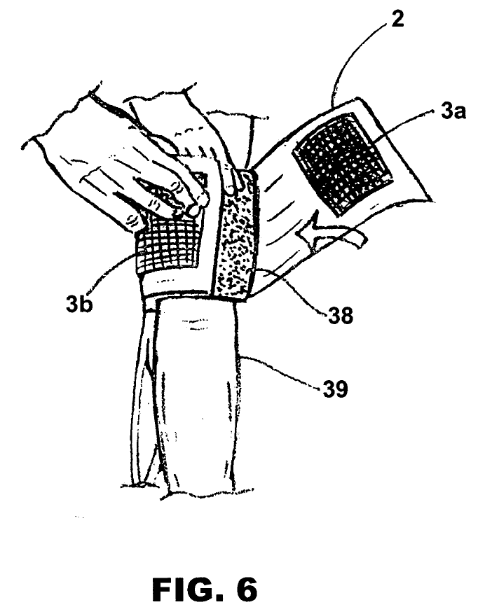

In the diagram in FIG. 5, an alternative embodiment is shown, where the sphygmomanometer includes a set of three chambers, each one of them to be used respectively according to the size and age of the patient. Indeed, in this embodiment three chambers are included, where the first chamber 32, the one with the longest range, is designed to be used with obese adults, a second chamber 33, with medium range, to be used with standard-sized adults, and a third chamber 34, with a lesser range than the previous ones, for general pediatric use. Chambers 32, 33 and 34 are connected to respective pairs of air conduits, respectively identified now by reference numbers 35, 36 and 37. In addition, preferably in this embodiment, chambers 32, 33 and 34 are an integral part of a single, indivisible structure and are separated and isolated one from the other by means of flexible materials making up partitions which separate the chambers. Thus, considering that the three chambers 32 to 34 as a whole define a single sleeve divided in three compartments or chambers by means of partitions, the sleeve shall perform the function of two, three or eventually more overlapping chambers should it be appropriate to add additional chambers. As it will be understood, if in practice it is deemed appropriate, all three chambers may as well be partitionable, one from the other as well as with respect to the clamping band 2. In addition, in this embodiment with partitioned chambers, two, three or more chambers may be used. In accordance with the number of chambers, a selector switch will be used, with the number of paths required in each case. In general, the width of the chambers may range, for example, from 3 to 18 cm or more, with several inter-mediate values depending on the type of patient, i.e., neonates, children, adults and obese adults.

The same FIG. 5 is useful for referring to the embodiments of the sphygmomanometer where the set of chambers is comprised by chambers arranged one inside the other, as well as when the chambers are divided in separate compartments which may be selected individually or together using the selector switch, in all cases to respond to operational needs ac-cording to the type of patient that is to be handled.

Indeed, for example, chamber 34 may be arranged inside chamber 33, or else chamber 33 inside chamber 32, or else chamber 34 inside chamber 33 and these two inside chamber 32. In addition, as it will be understood, the perimeter configuration, the surface defined by the contour of each chamber and the relative location may vary depending on practical considerations regarding the manufacture of the sphygmomanometer.

Regarding the other alternative embodiment, where chambers are divided in compartments separated by partitions, numerical references 32, 33 and 34 in this case must be construed as the compartments or chambers in which a single sleeve is divided and, thus, the lines delimiting the length and width of each pertain to partitions separating said compartments. Similarly to what was said regarding the annotated preceding alternative, both the geometrical configuration as well as the total extension or surface of each compartment and the relative location between them may vary depending on what is more convenient in practice from a technical point of view.

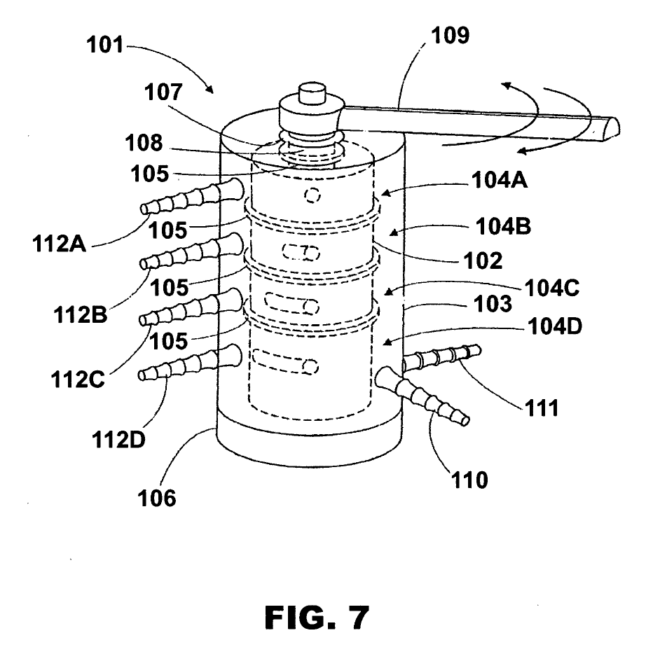

To use this sphygmomanometer and, in particular, when it takes, for example, the first preferred embodiment, initially the arm of the patient is wrapped with it or the chambers, depending on what is required for each type of patient, together with a stethoscope, while keeping clamping band 2 deployed, as shown in FIG. 7. If only one of the chambers is to be used, alternatively all chambers may be kept attached to band 2, or else those chambers which are not required at that point in time may be removed.

Next, the chamber(s) and the stethoscope are attached and wrapped with clamping band 2, whereupon all steps previous to the reading of the patient blood pressure are completed. In this FIG. 7, general reference number 38 is used to identify any single one of the chamber embodiments, i.e., independent, separable, partitioned, introsuspected or any combination of these different configurations, while reference number 39 is used to identify the arm of the patient, regardless of his age and size.

When put into practice, this sphygmomanometer may include optionally, a scale to select the insufflable chamber recommended to use, depending on the circle of the arm whose blood pressure is to be taken.

To be precise, this is a metric scale denominated in centimeters, although in practice any other type of scale may be used, such as international, calorimetric, etc., or measuring or weighting. Thus, the insufflable chamber to be used may be selected according to age, build and nutritional condition of the patient. This scale may be, for example, attached to the bracelet of the sphygmomanometer used, both in the case of a traditional sphygmomanometer as well as with the multipurpose sphygmomanometer object of this invention.

The incorporation of the scale to the chambers, made of cloth, vinyl or another type of support, may be by means of a tape with the relevant scale attached, stitched or fixed by any means, as well as by printing on the bracelet or the chamber, on its internal or external surface, in its edges or central areas.

Preferably, the scale used shall follow international practices. The insufflable chambers to be selected through the relevant paths shall have their match or equivalencies in the aforementioned scale. In this case, the scale is related to the coverage of approximately 80-100% of the brachial perimeter at the middle part of arm, as per international standards. A table follows which includes the measurements pertaining to chambers recommended according to the variables to be born in mind in each case.

For the values quoted in this table, the chambers defined by the classical insufflable chambers or their equivalent in chambers such as those of the multipurpose sphygmomanometer of this invention have been taken as reference.

| | | Chamber | Chamber | Width/length | Covering 80% to | | |
|---|---|---|---|---|---|---|---|
| Color | | width | length | Standardization | 100% of perimeter of | 100% Covering Range | Standardization Index |
| Scale | Age | [cm] | [cm] | Index | limb Range [cm] | 80% Covering Range | % of perimeter of limb |
| Yellow | New Born | 4 | 10 | 0.4 | 10 | 100%: up to 10 | 0.33 to 0.6 |
| | | | | | | 80%: up to 12 | 0.40 + 1/−7 |
| Green | Unweaned | 6 | 15 | 0.4 | 10.1 to 18 | 100%: up to 15 | 0.33 to 0.59 |
| | | | | | | 80%: up to 18 | 0.40 + 12/−7 |
| Orange | Child | 8 | 20 | 0.4 | 15.1 to 24 | 100%: up to 20 | 0.33 to 0.52 |
| | | | | | | 80%: up to 24 | 0.40 + 12/−7 |
| Violet | Adolescent | 10 | 25 | 0.4 | 20.1 to 30 | 100%: up to 25 | 0.33 to 0.49 |
| | Small Adult | | | | | 80%: up to 30 | 0.40 + 9/−7 |
| Brown | Adult | 12 | 30 | 0.4 | 25.1 to 36 | 100%: up to 30 | 0.33 to 0.47 |
| | | | | | | 80%: up to 36 | 0.40 + 9/−7 |
| Pink | Obese adult | 15 | 37.5 | 0.4 | 30.1 to 45 | 100%: up to 37 | 0.33 to 0.49 |
| | | | | | | 80%: up to 45 | 0.40 + 9/−7 |
| White | Very obese | 17 | 42.5 | 0.4 | 37.5 to 51 | 100%: up to 42 | 0.33 to 0.45 |
| | adult or thigh | | | | | 80%: up to 51 | 0.40 + 8/−7 |

The colors in this reference table coincide with the range of values highlighted with colors in the aforementioned scale for selection of the insufflable chamber recommended for use.

The operating procedure is summarized below.

The proper chamber or bracelet is placed around the arm of the patient and the scale is read, which scale may include colors to help speed up the relevant reading, and the location where point “0” or beginning of the scale coincides with the other point, or point of contact, is determined, and subsequently the scale is sufficiently adjusted to the arm, so that the choice of the proper chamber is more trustworthy.

If this point of contact is established, for example, in the “orange-colored” area, the sleeve corresponding to this color is chosen or selected, for example, the chamber for children, 13.9 cm long by 9 cm wide. In this case in particular, the scale includes seven colors from small to large (yellow, green, orange, violet, brown, pink and white). These colors relate to the different inflatable chambers with different measures to be used as already mentioned. The number of inflatable chambers and matching colors in the scale shall vary according to their number, depending on the number of utilities or tensiometer chambers.

It should be pointed out that the name of the insufflable chambers ac-cording to their ethereal name (RN, breast-fed babies, adults, etc.) does not necessarily coincide exactly in practice with the patients and, instead, are valid as mere guidelines for usage. That is why this scale indicates approximately which insufflable chamber is more adequate according to nutritional condition, age, size, etc. of the patient.

It becomes clear, then, that thanks to its outstanding structural features in any of its alternative embodiments, this tensiometer allows the use of chambers, individually or combined one with the other depending on each patient in particular, which is not possible with conventional tensiometers existing to this date.

In FIG. 7, a multi-way selector device 101 is shown; it comprises an outside body or carcass 103 inside which is housed the inside body or rotary drum 102, wherein a longitudinal attachment of fluid-channeling sectors 104A to 104D is defined, hermetically isolated by means of sealing rings 105. The carcass 103 is closed at the bottom by a cover 6, while at the top it has an opening 107 through which passes a cylindrical projection 108 of rotary drum 102 for coupling an actuating handle 109. Matching each one of said fluid-channeling sectors 104A to 4D of drum 102, carcass 103 is provided with orifices communicating with the outside wherein are coupled fluid inlet and outlet spouts where the respective hoses or cannulae are coupled.

According to this embodiment, the valve includes a fluid-injection spout 110, a spout 111 for connection to the manometer, and fluid-outlet spouts 112A to 112D for feeding and actuating the hydraulic or air-actuated devices the operation of which must be controlled by means of valve 101.

Depending on the position of actuating handle 109, and therefore the relative position of rotary drum 102 and carcass 103, the device 101 is kept closed or a selective and combined communication is established between the fluid inlet spout or simply inlet 110 and one or several of outlets 112D to 112D. In other words, in a first position of drum 102, the passage of fluid between inlet 110 and all outlets 112A to 112D is blocked, while at the four remaining positions successive communications are established between inlet 110 and each one of outlets 112A to 112D, with one of the outlets joining in each one of the positions.

As it will be understood, the sequence and combination in which the flow of fluid is enabled from inlet 110 to outlets 112A and 112D depends on the peculiarities of rotary drum 102 in its various possible forms of embodiment depending on what is more convenient in practice for each case of application of this valve. For instance, in the same manner as FIG. 7 shows that outlets 112A to 112D are aligned along carcass 103, these may likewise be arranged following an helicoidal geometry, aligned along the perimeter as a whole or in pairs on a single transversal plane, or even all of them arranged axially on the base of the valve body and, more precisely, the base of the carcass to which cover 106 is shown coupled.

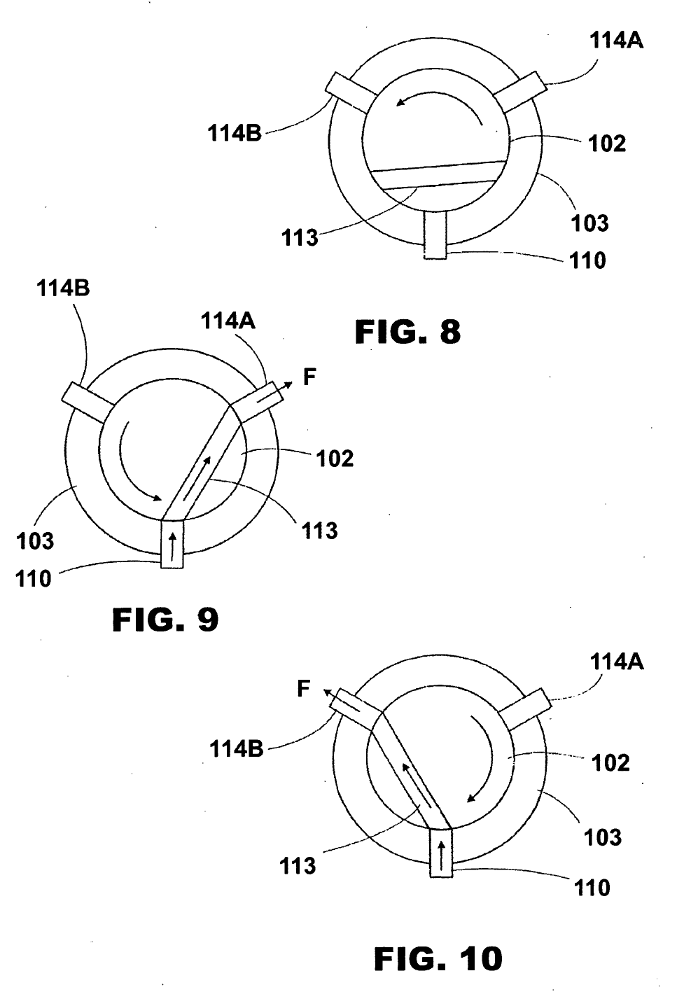

The diagrams in FIGS. 8 to 10, where the same reference numbers are kept for parts equivalent to those shown in FIG. 7, show a basic embodiment of the valve of this invention which may belong to any one of sectors 104A to 104D of same, or else a combination of two adjacent sectors. Starting with FIG. 8, drum 102 is shown to be provided with a channeling conduit 113 which stretches transversally following a geometrical chord defined on the circular section of said drum 102. As FIG. 8 shows, the relative position of drum 102 and carcass 103 corresponds to the “closed switch” condition, with inlet 110 cut off from the outlets now identified by means of references 114A and 114B. When drum 102 is turned to reach the position shown in FIG. 9, conduit 113 establishes communication between inlet 110 and outlet 114A, allowing fluid to go through as indicated by arrows F. In like manner, when drum 102 turns to reach the position illustrated in FIG. 10, communication is established between inlet 110 and outlet 114B. Thus, device 110 operates as a switching key between inlet 110 and one of the outlets 114A and 114B. If, as it has been mentioned, the illustration in these FIGS. 8 to 10 matches with some of the sectors of device 101 defined on the same transversal plane, one will have as many possible combinations of fluid outlets as sectors are formed in device 101.

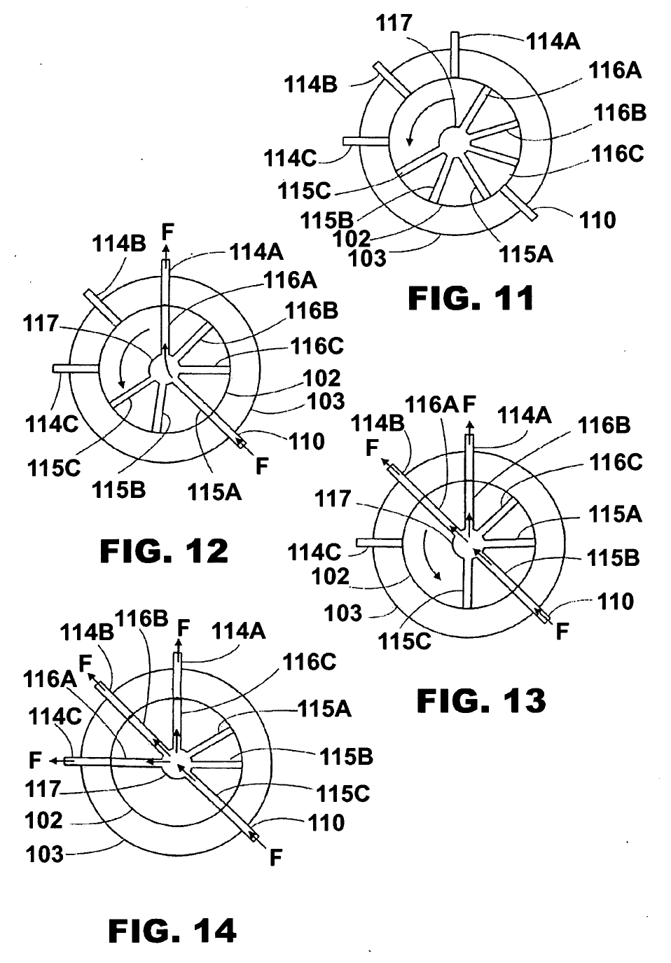

In FIGS. 11 to 14, another embodiment of device 101 is shown with, for instance, three fluid outlets aligned along the perimeter on a single transversal plane, where the rotary drum includes sections of channeling conduits which branch into sections of outlet conduits. The ends of each one of these sections of channeling conduits are selective and can be made to face the outlet orifices of device 101 in a selective and combinable manner in order to establish several paths of communication between fluid inlets and outlets. Indeed, drum 102 of device 101 now has formed a plurality of fluid channeling conduits which may be combined one with the other, forming two groups of conduits: On the one hand, inlet conduits 115A and 115D and, on the other, outlet conduits 116A to 116D, which project radially from a central chamber 117. When drum 102 is in the relative position to carcass 103 shown in FIG. 11, device 101 is kept closed, as fluid inlet 110 is not communicated with any of the inlet channeling conduits 115A to 115C. Instead, when drum 102 is turned to the position shown in FIG. 12, conduit 115A is faced with inlet 110 and, consequently, fluid circulation is enabled towards outlet 114A through conduit 115A, central chamber 117 and outlet conduit 116A. When drum 102 is turned to the position illustrated in FIG. 13, inlet conduit 115B is faced with inlet 110, outlet conduit 116A is faced with outlet 114B and outlet conduit 116B is faced with outlet 114A and, in this manner, fluid circulation from inlet 110 to outlets 114A and 114B is enabled. Finally, as shown in FIG. 14, when the drum is placed so as to face inlet conduit 115C at fluid inlet 110, outlet conduits 116A, 116B and 116C are faced, respectively, with outlets 114C, 114B and 114A, with fluid circulating now from inlet 110 simultaneously to the three outlets aforementioned. As it will be understood, the number of fluid-inlet and fluid-outlet channeling conduits of rotary drum 102, in practice, may vary depending on the number of fluid-outlets required in particular in each case this device 101 is applied.

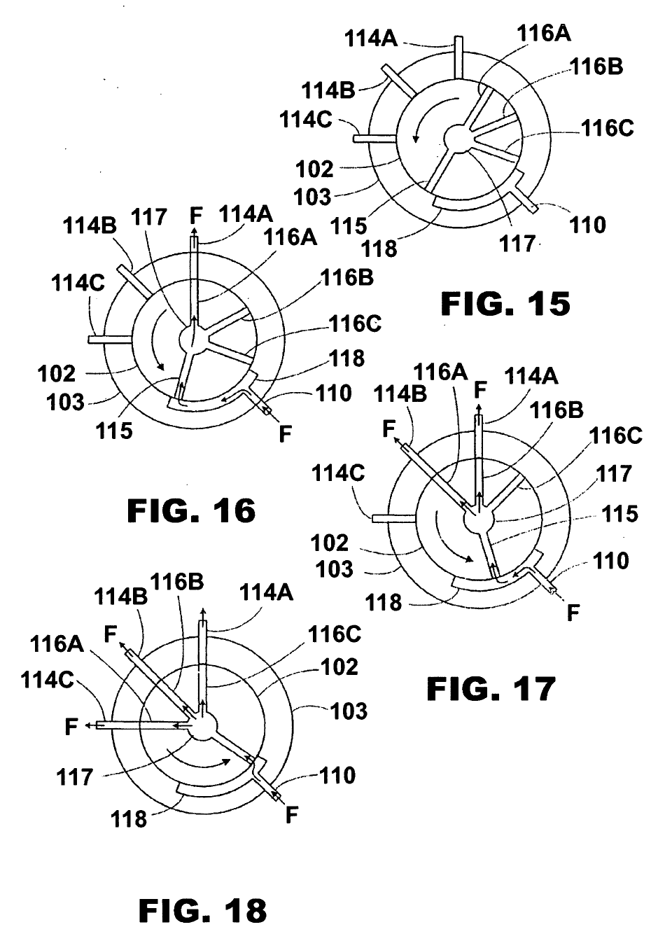

In FIGS. 15 to 18, an embodiment is shown of device 101, the parts of which equivalent to those already described are identified by means of the same reference numbers, where the rotary drum includes a fluid-inlet conduit which branches into three outlet conduits and where the ends of each one of said channeling conduits likewise and may be selected and faced jointly with the respective outlets of device 101. However, in this case the inside surface of carcass 103 has a pre-chamber 118 in the shape of a circle arc which allows selective communication of fluid inlet 10 with the channeling conduit of inlet 115 and one or more of inlets 114A to 114C through central chamber 117 and conduits 116A to 116C. The extension of said pre-chamber is at least equal to the distance between the adjacent ends of conduits 116A to 116C which face outlets 114C to 114C. Thus, it may be said that pre-chamber 118 allows the replacement of two of the three inlet conduits 115A to 115C of the embodiment shown in FIGS. 11 to 14. In addition, pre-chamber 118 may eventually extend also along drum 102 so as to intercommunicate two or more sectors of device 101 in each of which one or more fluid outlets is formed. Thus, the diagrams shown in these figures may relate to one of the various sectors of device 101, defined in respective transversal planes of the body thereof.

In this way, as device 101 has only one fluid inlet 110, pre-chamber 118 enables communication between said inlet 110 and the respective fluid outlets pertaining to each one of those sectors. It is readily evident that pre-chamber 118 may take in practice several shapes depending on he number of fluid outlets which must be arranged in each case of application of device 101.

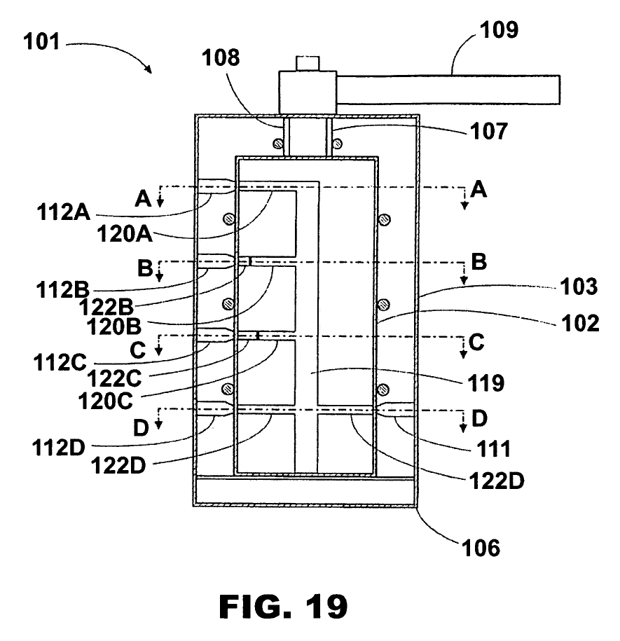

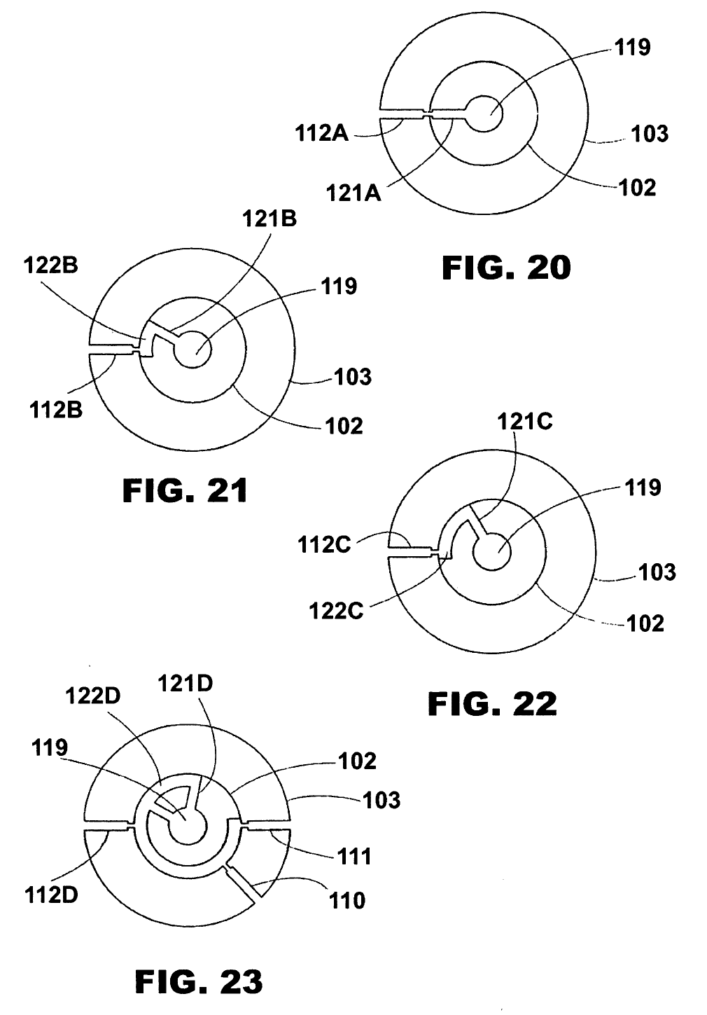

The elevated and partially sectional view of device 101 which is shown in FIG. 19 and which relates essentially to the embodiment illustrated in FIG. 7, highlights in addition some of the peculiarities of drum 102 in relation to carcass 103 in a position which establishes communication between fluid inlet 110 and outlets 112A and 112D; this may be under-stood more clearly in the diagrams of FIGS. 20 to 23. It should be pointed out, for purposes of simplifying the illustrations, that although in FIG. 7 references 112A to 112D have been used to identify fluid outlet spouts, now, the same reference numbers indicate directly the fluid outlet orifices formed in carcass 103.

In like manner, as was the case in the figures already described, now fluid inlet 110 and the connection of manometer 111 match spouts 115 and 111 in FIG. 7. In this FIG. 19 one can see that rotary drum 102 includes a longitudinal chamber 119 from which a succession of outlet conduits 120A to 120C, which relate respectively to each one of outlets 112A to 112C of the sectors defined according to transversal planes A-A to C-C, projects radially. Pre-chambers 122B to 122D, the configuration of which will be clearly shown in the following figure, are likewise depicted.

Indeed, FIGS. 20 to 23 show, between rotary drum 102 and carcass 103, matching transversal planes B-B to C-C, the configuration of respective pre-chambers 122B to 122D in the shape of a circle arc, which complete the selective communication of outlets 112B to 112D with fluid inlet 110 and the connection of manometer 111 through chamber 119 and conduits 121B to 121D. As shown, said pre-chambers 122B to 122D are of different lengths in each of the transversal planes, are defined in partly overlapping positions according to planes which are perpendicular to same, so that a selective and combined communication between any of the matching channeling conduits may be established and, consequently, with any of inlets 112B to 112D, with fluid inlet 110 and manometer connection 111. According to optional alternative embodiment options, pre-chambers 122B to 122D may also be formed in carcass 103 or else may include an arc portion formed on the rotary drum 102 and another portion formed in carcass 103. The selection of fluid outlets, for example, identified by means of reference numbers 112A to 112D will depend on chambers 4 5 to be used with the sphygmomanometer, which may be partitioned, introsuspected, overlapping, detachable, etc.

In the case of introsuspected or overlapping chambers, the fluid out-lets used shall be equivalent to the number of inflatable chambers. For ex-ample, five different sleeves may be actuated sequentially according to the needs of usage turning the rotary drum 102 by means of handle 109.

In the case of partitions or equivalent chambers, actuation of same shall be sequential, and adding from small to large in general, depending on the need to use it for neonates, children, adults of obese subjects, or any other intermediate patient.

In each case, the variant shall relate in the main to the configuration of the conduits of rotary drum 102 which, instead of having a single communicating conduit enables to operate sequentially, shall have several to operate in sequence and addition, with each one communicated with a set number of chambers, equivalent to the number and configuration of conduits available in the rotary drum.

Moreover, as an outlet is selected and others are enabled, different equivalences to conventional insufflable chamber of different sizes shall be achieved.

What is claimed is:

1. A sphygmomanometer device for measuring the blood pressure of various sized patients, including in combination:
a flexible bracelet structurally arranged to be wrapped about a patient's jointed appendage;
a plurality of insufflable chambers associated with said flexible bracelet, with at least one of said insufflable chambers being structurally arranged to predeterminately engage the appendage of a various sized patient when said bracelet is wrapped about the patient's appendage;
a means of insufflation operatively connected to said plurality of insufflable chambers to generate a pressure therein;
a manometer selectively connected to each of said insufflable chambers to provide an indication of the patients' blood pressure; and
enabling means structurally arranged to establish communication between said means of insufflation and said plurality of insufflable chambers to generate a pressure therein.

2. The sphygmomanometer device in accordance with claim 1, wherein said plurality of insufflable chambers are defined by partitioning of said bracelet.

3. The sphygmomanometer device in accordance with claim 1, wherein said plurality of insufflable chambers are housed inside a receptacle shaped by said bracelet.

4. The sphygmomanometer device in accordance with claim 1, wherein said insufflable chambers are attached to said bracelet.

5. The sphygmomanometer device in accordance with claim 1, wherein said insufflable chambers are of varying lengths and housed one inside the other.

6. The sphygmomanometer device in accordance with claim 1, wherein said bracelet includes a supplementary bracelet which extends said bracelet and wherein said supplementary bracelet includes a supplementary insufflable chamber which may be connected to said selective enabling means.

7. The sphygmomanometer device in accordance with claim 1, wherein the device includes means of identification of said insufflable chambers for their operative coupling to said selective enabling means.

8. The sphygmomanometer device in accordance with claim 7, wherein said means of identification of the chambers for their operative coupling are located in said bracelet and in said selective enabling means.

9. The sphygmomanometer device in accordance with claim 7, wherein said means to identify said insufflable chambers relates to a set value in the scale of values table.

10. The sphygmomanometer device in accordance with claim 7, wherein said means to identify said chambers include colors.

11. The sphygmomanometer device in accordance with claim 7, wherein said means to identify said chambers include alphabetical and/or numeric signs.

12. The sphygmomanometer device in accordance with claim 7, wherein said means to identify said chambers include measuring scales.

13. The sphygmomanometer device in accordance with claim 7, wherein said means to identify said chambers include high and/or bass relief signs.

14. The sphygmomanometer device in accordance with claim 1, wherein said means of enabling includes a valve with at least two exit ways.

15. The sphygmomanometer device in accordance with claim 1, wherein said means of selective enabling include a valve means of selective enabling means to discharge insufflated air.

16. The sphygmomanometer device in accordance with claim 1, wherein said means of enabling includes a fluid selector device made up of two attached bodies having rotating capacity one in relation to the other and provided with respective fluid inlet and outlet openings located in a different angular position and the selective confrontation of which through rotation, enables the alternative communication between said means of insufflation and said separate insufflable chambers.

17. The sphygmomanometer device in accordance with claim 16, wherein said fluid selector device comprises an outside body in the inside housing of which is wedged an inside cylindrical body with rotary capability one in relation to the other.

18. The sphygmomanometer device in accordance with claim 16, wherein said fluid selector device includes an outside body provided with at least one inlet connectable to said means of insufflation and a set of outlets connectable to separate insufflable chambers.

19. The sphygmomanometer device in accordance with claim 17, wherein in said fluid selector device said inside body is provided with mobile communication conduits, the selective positioning of which is capable of communicating the fluid inlet or inlets to one or more outlets, individually or together.

20. The sphygmomanometer device in accordance with claim 19, wherein said mobile communication conduits cross the inside body.

21. The sphygmomanometer device in accordance with claim 19, wherein said fluid selector device includes surface communication conduits which extend associated openings.

22. The sphygmomanometer device in accordance with claim 19, wherein said inside body includes longitudinal and transverse conduits.
